(19) United States
(12) Patent Application Publication
(10) Pub. No.: US 2026/0241118 A1
(43) Pub. Date: Aug. 20, 2026

(54) TWO-DOSE DOSAGE SYSTEM FOR FLUID PRODUCT DISPENSING DEVICE

(71) Applicant: APTAR FRANCE SAS, Le Neubourg (FR)

(72) Inventor: Sébastien MICHAUX, Le Petit Quevilly (FR)

(73) Assignee: APTAR FRANCE SAS, Le Neubourg (FR)

(21) Appl. No.: 19/155,688

(22) PCT Filed: Feb. 12, 2024
(86) PCT No.: PCT/FR2024/050185

§ 371 Date: Aug. 12, 2025

(30) Foreign Application Priority Data
Feb. 13, 2023 (FR) FR2301329

(51) Int. Cl. A61M 15/00; A61M 11/00; A61M 15/08; G01F 11/02

(52) U.S. Cl. CPC A61M 15/0065; A61M 11/007; A61M 15/009; A61M 15/08; G01F 11/027; A61M 2210/0618

(57) ABSTRACT

Two-dose dosage system for fluid product dispensing device, the device having a syringe-type reservoir containing fluid product, a piston secured to a piston rod being slidably mounted in the reservoir to dispense the fluid product. The dosage system having a first dosage element, a second dosage element and a third dosage element, the first dosage element being fixed in an axially non-movable manner on said piston rod, such that it moves axially together with it, the third dosage element being axially non-movable relative to the reservoir, the second dosage element being arranged between the first and third dosage elements, the second dosage element sliding relative to the third dosage element during the dispensing of the first dose and relative to the first dosage element during the dispensing of the second dose.

The present invention relates to a two-dose dosage system and a fluid product dispensing device comprising such a two-dose dosage system.

Fluid product dispensing devices of the two-dose type are well known. They comprise a reservoir containing two doses of fluid product to be dispensed in two successive actuations. When the reservoir is of the syringe-type, with a piston secured to a piston rod which slides in a cylindrical reservoir, two-dose dosage systems exist which allow to separate the stroke of the piston into two half-strokes, one for each dose. These dosage systems are often complex and therefore, expensive to manufacture and to assemble, and generally require a modification of the syringe and/or of the piston rod.

Furthermore, when a nasal spray head is associated with a syringe-type reservoir, a disadvantage of the existing devices relates to the quality of the spray generated, which may depend on the actuation carried out by the user, particularly the speed and the actuation force, and may differ between the two doses. However, to guarantee a good reproducibility of the spray, it is desirable to generate a calibrated spray, i.e. the properties of which are substantially similar for the two doses, independently of the way in which the user actuates the device.

Documents U.S. Pat. No. 10,792,433, AU2013238598 and US2019060578 describe devices of the prior art.

The present invention aims to provide a two-dose dosage system and a fluid product dispensing device which does not reproduce the above-mentioned disadvantages.

The present invention aims particularly, to provide a two-dose dosage system which adapts to a fluid product dispensing device comprising a standard syringe-type reservoir, in particular, without having to modify the reservoir and/or the piston rod.

The present invention also aims to provide a two-dose dosage system and a fluid product dispensing device which allows to ensure a calibrated and reproducible spray for both doses, in particular, independently of the way in which the user actuates the device.

The present invention also aims to provide a two-dose dosage system and a fluid product dispensing device, which guarantee a precise dosage for both doses.

The present invention also aims to provide a two-dose dosage system and a fluid product dispensing device, which are simple and inexpensive to manufacture and to assemble.

The present invention therefore, provides a two-dose dosage system for a fluid product dispensing device, said device comprising a syringe-type reservoir containing fluid product, a piston secured to a piston rod being slidably mounted in said reservoir to dispense said fluid product, said dosage system comprising a first dosage element, a second dosage element and a third dosage element, said first dosage element being fixed in an axially non-movable manner on said piston rod, so that it moves axially together with it, said third dosage element being axially non-movable relative to said reservoir, said second dosage element being arranged between said first and third dosage elements, said second dosage element sliding relative to said third dosage element during dispensing of the first dose and relative to said first dosage element during dispensing of the second dose.

Advantageously, each dosage element is C-shaped, with an axial opening along its entire height allowing it to be positioned around said piston rod.

Advantageously, said piston rod comprises at its rear axial end edge, a bearing flange on which the user presses during the actuation, said first dosage element comprising a housing adapted to be fixed on said bearing flange.

Advantageously, said reservoir is formed by a hollow cylinder provided with a collar at its rear axial end edge, said third dosage element comprising a front axial edge which, in use, bears on said collar.

Advantageously, said first and third dosage elements have approximately identical inner and/or outer diameters, said second dosage element having an outer diameter less than the inner diameter of said first and third dosage elements, such that said second dosage element slides inside said third dosage element during the dispensing of the first dose and inside said first dosage element during the dispensing of the second dose.

Advantageously, said third dosage element is removed from said piston rod after dispensing the first dose, to allow the second dose to be dispensed.

Advantageously, said second dosage element is connected to said third dosage element by first resistance means and to said first dosage element by second resistance means.

Advantageously, said first resistance means generate a lower resistance than said second resistance means, such that during the actuation, it is firstly, said first resistance means that are overcome to dispense the first dose.

According to a first advantageous embodiment, said first, second and third dosage elements are formed by three separate parts assembled together.

Advantageously, said first resistance means comprise an external radial projection of said second dosage element, extending radially outwards, which cooperates with an inner groove formed in said third dosage element.

Advantageously, said external radial projection is formed at a front axial edge of said second dosage element, and said inner groove is formed near a rear axial edge of said third dosage element, being defined between said rear axial edge and an inner radial projection of said third dosage element.

In a second advantageous embodiment, said first, second and third dosage elements are moulded of a single one-piece.

Advantageously, said first resistance means are formed by one or more first breakable bridges, and said second resistance means are formed by one or more second breakable bridges.

Advantageously, said first and third dosage elements are connected to one another in a captive manner, particularly via a wire.

Advantageously, the first dose is defined by the axial movement of said first dosage element, axially fixed relative to said piston rod, relative to said third dosage element, axially fixed relative to said reservoir.

Advantageously, the second dose is defined by the axial movement of said first dosage element, axially fixed relative to said piston rod, relative to said second dosage element, when it is axially fixed relative to said reservoir.

The present invention also provides a fluid product dispensing device comprising a syringe-type reservoir, a piston slidably mounted in said reservoir, said piston being secured to a piston rod, and a two-dose dosage system as described above, said dosage system being assembled on said piston rod.

Advantageously, the device comprises a nasal spraying head provided with a spraying orifice mounted on said reservoir.

These advantages and characteristics and others of the present invention will appear more clearly during the detailed description below, made in reference to the accompanying drawings, given as non-limiting examples, and in which:

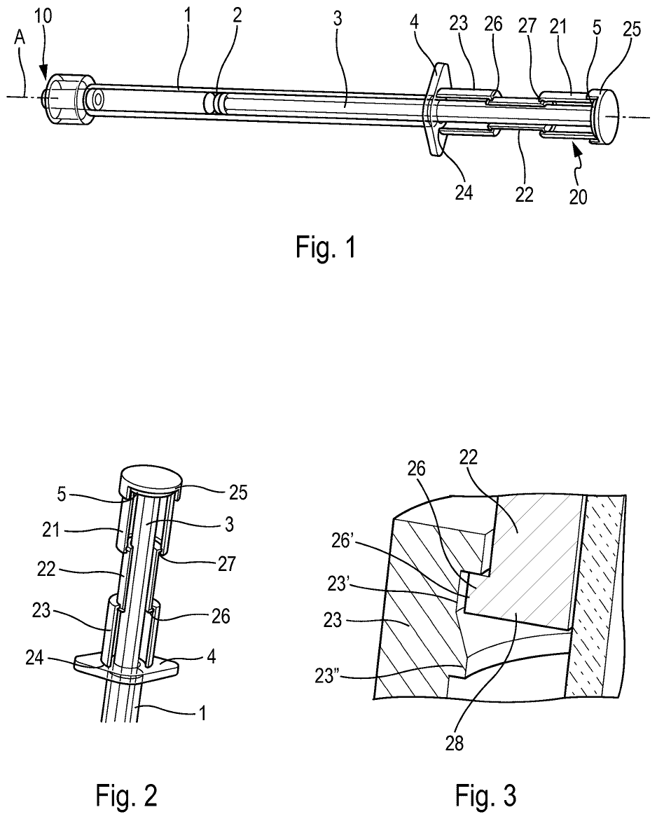

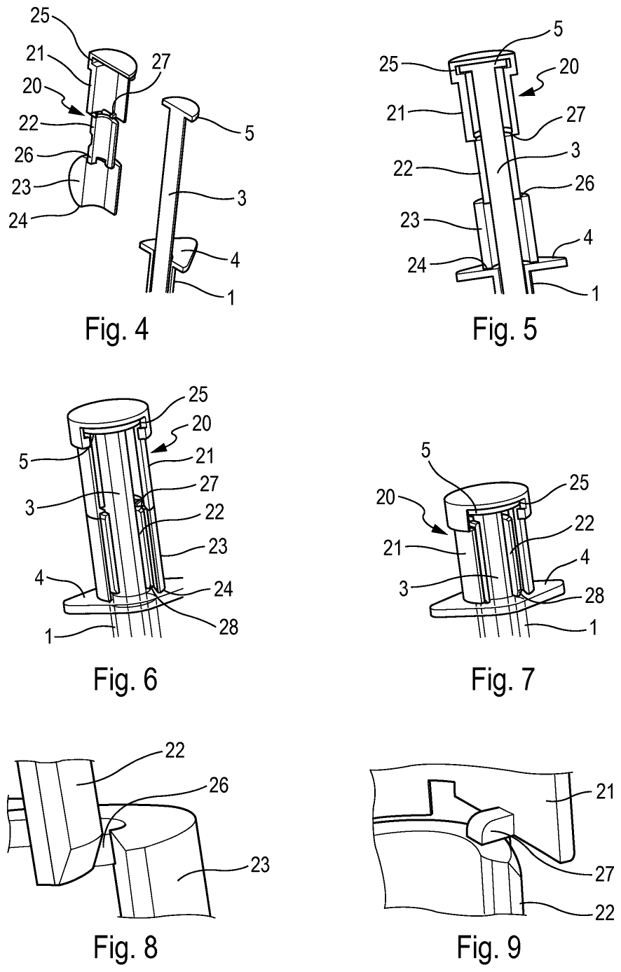

FIG. 1 is a schematic perspective view of a fluid product dispensing device comprising a two-dose dosage system according to a first embodiment, in a rest position,

FIG. 2 is a view of detail of FIG. 1, showing the two-dose dosage system,

FIG. 3 is a view of detail of FIG. 2, showing the first resistance means,

FIG. 4 is a partial schematic exploded perspective view of a fluid product dispensing device comprising a two-dose dosage system according to a second embodiment, before assembly of the two-dose dosage system on the piston rod of the device,

FIG. 5 is a view similar to that of FIG. 4, after assembly of the two-dose dosage system on the piston rod and before the first actuation.

FIG. 6 is a view similar to that of FIG. 5, after dispensing the first dose,

FIG. 7 is a view similar to that of FIG. 6, after dispensing the second dose,

FIG. 8 is a detailed schematic perspective view, illustrating a first breakable bridge forming first resistance means for the first dose,

FIG. 9 is a detailed schematic perspective view, illustrating a second breakable bridge forming second resistance means for the second dose,

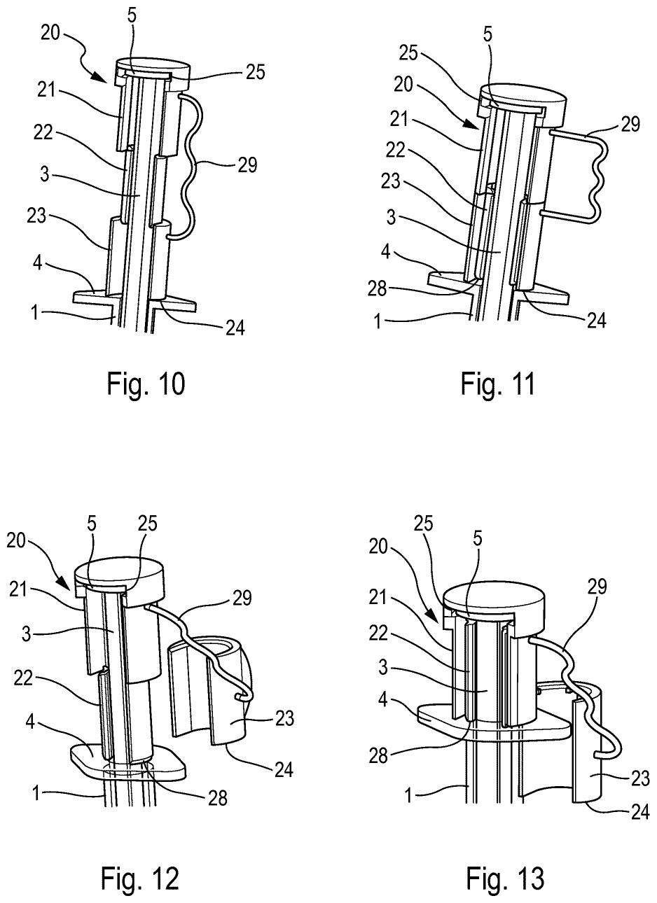

FIG. 10 is a view similar to that of FIG. 5, showing a variant embodiment of the second embodiment, and

FIGS. 11 to 13 are views similar to that of the FIG. 10, showing respectively the positions after dispensing the first dose, before and after dispensing the second dose.

The terms “axial” and “radial” refer to the longitudinal central axis A of the dispensing device, shown in FIG. 1. The terms “front” and “rear” refer to the direction of flow of the fluid product during the actuation of the device.

The present invention will be described below with reference to embodiments regarding to fluid product dispensing devices provided with a spray head, but it is understood that other embodiments could be envisaged, particularly, conventional injection syringes, comprising a needle for dispensing the fluid product.

FIG. 1 very schematically represents a spray head 10 assembled on a syringe-type reservoir 1. A removable protective cap may be assembled on the spray head before using the device. The reservoir 1 contains two doses of fluid product, generally a liquid, which are dispensed in two successive actuations.

Conventionally, suitable dispensing means are provided in the reservoir 1 to dispense its contents. Typically, these dispensing means comprise a piston 2 slidably mounted in the reservoir 1 and secured to a piston rod 3 actuated by the user at the time of the actuation.

The reservoir 1 is formed by a hollow cylinder provided with a collar 4 at its rear axial end edge. The piston rod 3 comprises, at its rear axial end edge, a bearing flange 5 on which the user presses during the actuation.

The spray head 10 can conventionally comprise a body provided with a spraying orifice. Preferably, the spray head 10 is of the nasal type, with an extended body to penetrate into a nostril and the spraying orifice arranged at the front axial end of said body, in the flow direction of the fluid product during the actuation. Advantageously, an insert can be arranged upstream of the spraying orifice to generate a spray, for example by means of a well-known spraying profile. Other types of spry head are also considered.

According to the invention, a two-dose dosage system 20 is provided which is fixed to the piston rod 3, between the collar 4 of the reservoir 1 and the bearing flange 5 of the piston rod 3.

This dosage system 20 comprises a first dosage element 21, a second dosage element 22 and a third dosage element 23. Each dosage element 21, 22, 23 is advantageously C-shaped, comprising an axial opening along its entire height allowing it to be positioned around the piston rod 3.

The first dosage element 21 is fixed in an axially non-movable manner on the piston rod 3, such that it moves together with it. Advantageously, it comprises a housing 25 adapted to receive the bearing flange 5 of the piston rod 3.

The third dosage element 23 is axially non-movable relative to the reservoir 1. Advantageously, it comprises a front axial edge 24 which, in use, bears on the collar 4 of the syringe-shaped reservoir 1.

Thus, when the dosage system 20 is arranged on the piston rod 3 of the dispensing device, the position of the piston 2 in the reservoir 1 is determined with precision and reproducibly, since the dosage system 20 bears, on the one hand, on the collar 4 of the reservoir 1 and, on the other hand, receives the bearing flange 5 of the piston rod 3.

The second dosage element 22 is arranged between the first and third dosage elements 21, 23.

Advantageously, the second dosage element 22 is connected to the third dosage element 23 by first resistance means 26 and to the first dosage element 21 by second resistance means 27. These first and second resistance means 26, 27 allow to accumulate energy in the user's hand during the actuation, which is suddenly released when they are overcome, which guarantees good dispensing of the entirety of each dose.

The first resistance means 26 generate a lower resistance than the second resistance means 27, such that during the actuation, it is firstly the first resistance means 26 that are overcome to dispense the first dose.

The first and third dosage elements 21, 23 have approximately identical inner and/or outer diameters, while the second dosage element 22 has an outside diameter less than the inside diameter of the first and third dosage elements 21, 23. Thus, the second dosage element 22 can slide inside the third dosage element 23 during the dispensing of the first dose and inside the first dosage element 21 during the dispensing of the second dose.

Thus, at the start of the actuation, the first resistance means 26 are overcome when the axial force exerted by the user on the piston rod 3 reaches a first predetermined threshold, such that the unit formed by the first dosage element 21 and the second dosage element 22 slides relative to the third dosage element 23. FIGS. 1, 2, 5 and 10 illustrate the position before the first actuation and FIGS. 6 and 11 the position after dispensing the first dose.

The the first and third dosage elements 21, 23 having an approximately identical diameters, the actuation end of stroke for the first dose is defined by the abutment of the front axial edge of the first dosage element 21 on the rear axial edge of the third dosage element 23, as can be seen in FIGS. 6 and 11. The first dose is therefore, defined by the axial movement of the first dosage element 21, axially fixed relative to the piston rod 3, relative to the third dosage element 23, axially fixed relative to the reservoir 1.

In addition, in the position after dispensing of the first dose, the front axial edge 28 of the second dosage element 22 is in abutment on the collar 4 of the reservoir 1, as is the front axial edge 24 of the third dosage element 23.

To be able to dispense the second dose, the user must remove the third dosage element 23 from the piston rod 3, as shown in FIG. 12.

When the user presses the piston rod 3 again, the second resistance means 27 are overcome when the axial force exerted by the user on the piston rod 3 reaches a second predetermined threshold, such that the first dosage element 21 slides around the second dosage element 22. The actuation end of stroke for the second dose is defined by the abutment of the front edge of the first dosage element 21 on the collar 4 of the reservoir 1, as can be seen in FIGS. 7 and 13.

The axial lengths of the dosage elements 21, 22, 23 determine the volumes of the two doses. Thus, by varying these lengths, it is possible to adjust these volumes.

Preferably, the two doses have the same volume, and in this case, the dosage elements 21, 22, 23 have substantially identical lengths. Alternatively, it is possible to achieve two doses of different volumes by achieving dosage elements 21, 22, 23 of different lengths.

With reference to FIGS. 1 to 3, a first advantageous embodiment of the two-dose dosage system 20 is shown, in which the first, second and third dosage elements 21, 22, 23 are formed by three separate parts assembled to each other.

FIG. 3 shows an example for the first resistance means 26. In this example, an external radial projection 26′ of the second dosage element 22, extending radially outwards, cooperates with an inner groove 23′ formed in the third dosage element 23.

The external radial projection 26′ is formed at the front axial edge 28 of the second dosage element 22.

The inner groove 23′ is formed close to the rear axial edge of the third dosage element 23, by being defined between said rear axial edge and an inner radial projection 23″ of said third dosage element 23.

Thus, to overcome the first resistance means 26, the external radial projection 26′ of the second dosage element 22 must deform and pass over the inner radial projection 23″ of the third dosage element 23.

Advantageously, the second resistance means 27 are made in a similar manner, however, with a higher resistance requiring the application of a greater force to be overcome relative to the first resistance means 26.

FIGS. 4 to 9 show a second embodiment, in which the first, second and third dosage elements 21, 22, 23 are moulded of a single one-piece. In this case, the first and second resistance means 26, 27 which are advantageously formed by one or more first and second breakable bridges.

FIG. 8 shows an example of a first breakable bridge forming the first resistance means 26, arranged between the second and third dosage elements 22, 23.

FIG. 9 shows an example of a second breakable bridge forming the second resistance means 27, arranged between the first and second dosage elements 21, 22.

Preferably, the force required to break the first breakable bridge(s) is less than that required to break the second breakable bridge(s).

FIGS. 10 to 13 illustrate a variant of the second embodiment of FIGS. 4 to 9, in which the third dosage element 23 is connected in a captive manner to the first dosage element 21, for example by a wire 29. It should be observed that this variant could also be applied to the first embodiment in FIGS. 1 to 3. The wire 29 can be co-moulded, overmoulded, or fixed in any suitable manner to the first and third dosage elements 21, 23.

Although the present invention has been described above with reference to several embodiments thereof, it is understood that the invention is not limited to the embodiments described and represented in the drawings, but, on the contrary, any useful modification could be applied thereto by a person skilled in the art, without going beyond the scope of the present invention, such as defined by the accompanying claims.

1. A two-dose dosage system for a fluid product dispensing device, said device comprising a syringe-type reservoir containing fluid product, a piston secured to a piston rod being slidably mounted in said reservoir to dispense said fluid, characterised in that said dosage system comprises a first dosage element, a second dosage element and a third dosage element, said first dosage element being fixed in an axially non-movable manner on said piston rod, such that it moves axially together with it, said third dosage element being axially non-movable relative to said reservoir, said second dosage element being arranged between said first and third dosage elements, said second dosage element sliding relative to said third dosage element during the dispensing of the first dose and relative to said first dosage element during the dispensing of the second dose.

2. The system according to claim 1, wherein each dosage element is C-shaped, with an axial opening along its entire height allowing it to be positioned around said piston rod.

3. The system according to claim 1, wherein said piston rod comprises at its rear axial end edge a bearing flange on which the user presses during the actuation, said first dosage element comprising a housing adapted to be fixed on said bearing flange.

4. The system according to claim 1, wherein said reservoir is formed by a hollow cylinder provided with a collar at its rear axial end edge, said third dosage element comprising a front axial edge which, in use, bears on said collar.

5. The system according to claim 1, wherein said first and third dosage elements have approximately identical inner and/or outer diameters, said second dosage element having an outer diameter less than the inner diameter of said first and third dosage elements, such that said second dosage element slides inside said third dosage element during the dispensing of the first dose and inside said first dosage element during the dispensing of the second dose.

6. The system according to claim 1, wherein said third dosage element is removed from said piston rod after dispensing the first dose, to allow the dispensing of the second dose.

7. The system according to claim 1, wherein said second dosage element is connected to said third dosage element by first resistance means and to said first dosage element by second resistance means.

8. The system according to claim 7, wherein said first resistance means generate a resistance lower than said second resistance means, such that during the actuation, it is firstly said first resistance means that are overcome to dispense the first dose.

9. The system according to claim 7, wherein said first, second and third dosage elements are formed by three separate parts assembled together.

10. The system according to claim 9, wherein said first resistance means comprise an external radial projection of said second dosage element, extending radially outwards, which cooperates with an inner groove formed in said third dosage element.

11. The system according to claim 10, wherein said external radial projection is formed at a front axial edge of said second dosage element, and said inner groove is formed near a rear axial edge of said third dosage element, by being defined between said rear axial edge and an inner radial projection of said third dosage element.

12. The system according to claim 7, wherein said first, second and third dosage elements are moulded of a single one-piece.

13. The system according to claim 12, wherein said first resistance means are formed by one or more first breakable bridge(s), and said second resistance means are formed by one or more second breakable bridge(s).

14. The system according to claim 1, wherein said first and third dosage elements are connected to one another in a captive manner, particularly, via a wire.

15. The system according to claim 1, wherein the first dose is defined by the axial movement of said first dose element, axially fixed relative to said piston rod, relative to said third dose element, axially fixed relative to said reservoir.

16. The system according to claim 1, wherein the second dose is defined by the axial movement of said first dosage element, axially fixed relative to said piston rod, relative to said second dosage element, when it is axially fixed relative to said reservoir.

17. A fluid product dispensing device comprising a syringe-type reservoir, a piston slidably mounted in said reservoir, said piston being secured to a piston rod, and a two-dose dosage system according to claim 1, said dosage system being assembled on said piston rod.

18. The device according to claim 17, comprising a nasal spray head provided with a spraying orifice mounted on said reservoir.
